(19) United States
(12) Patent Application Publication
(10) Pub. No.: US 2009/0117162 A1
(43) Pub. Date: May 7, 2009

(54) Topical Cosmetic Compositions

(76) Inventors: Bruce H. Victor, Clifton, NJ (US); Kevin N. DiNicola, Wolcott, CT (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/261,110

(22) Filed: Oct. 30, 2008

Related U.S. Application Data
(60) Provisional application No. 61/001,415, filed on Nov. 1, 2007.

(51) Int. Cl. A61K 8/72; A61K 8/87; A61Q 19/00

(52) U.S. Cl. 424/401

(57) ABSTRACT

Disclosed is a cosmetic composition for topical application to the skin. The cosmetic composition comprises micron-sized, polymer-coated glass ball lenses having a polymer coating on the exterior surface thereof. The polymer-coated glass ball lenses are enhanced by engineered illumination and optics, and the appearance of the skin to which the cosmetic is applied is observable without masking the underlying skin.

### CROSS-REFERENCE TO RELATED APPLICATIONS

This application claims the benefit of U.S. Provisional Application No. 61/001,415, filed Nov. 1, 2007, the disclosure of which is incorporated herein by reference in its entirety.

### FIELD OF THE INVENTION

The present invention relates to cosmetic compositions for topical application to the skin. The cosmetic compositions contain ball lenses comprising micron-sized, polymer-coated glass ball lenses. The polymer-coated glass ball lenses enhance the appearance of the skin to which the cosmetic is applied without masking the underlying skin.

### BACKGROUND OF THE INVENTION

Heretofore, a characteristic method of concealing imperfections in the skin has been to apply makeup that is essentially opaque. The opaque material serves to cover blemishes, flaws, or other skin imperfections appearing on the skin, thus hiding such skin defects from optical view. The types of makeup or cosmetic compositions that have been previously employed typically contain high levels of metal oxides which serve to provide an effective invisible barrier concealing the flaw lying beneath the makeup. One drawback associated with the use of such opaque makeup is that it typically needs to be applied in rather thick and heavy coatings. Often users of the makeup find this objectionable and thus generally undesirable.

Recently, the cosmetic industry has sought to develop makeup compositions that need not be applied as thick and heavy masks, but instead which reflect light in a certain manner so as to prevent the observer's eye from seeing flaws or blemishes that may exist on the surface of the skin. One such approach is disclosed in US Patent Application Publication US2004/0120908A1 to Cohen et al. published on Jun. 24, 2004. This approach utilizes a topical application to the skin comprising a transparent component and a “non-interference” platelet component having specified light transmission and light reflectance. The transparent component comprises glass spheres or beads which essentially act as the light transmitting portions of a typical two-way mirror. The platelet component, such as an alumina flake, serves as an optical barrier to conceal flaws or blemishes.

U.S. Pat. No. 4,764,424 to Atochem discloses glass particles or beads that are coated with a polyamide or nylon layer to protect the particles or beads or to bind them to other materials using melting and cooling. In cosmetics applications, such a polyamide or nylon layer would typically be opaque or translucent, thus providing an optical barrier.

U.S. Pat. Nos. 5,830,485, 6,123,951 and 6,333,043 to L'Oreal disclose colored cosmetic compositions comprising a particular filler and a colorant wherein at least a portion of the filler is coated with a polymer containing a colorant. The particular filler may be selected from a mixture of both organic and inorganic materials including glass beads having a particle size upwards of 180 microns, for example. Cosmetic compositions containing such large size particles would be visible to the naked eye and objectionable to the consumer.

Published U.S. Patent Application Nos. 20050031558 and 20050276774 to Ciba disclose cosmetic compositions that contain a blend of at least two microencapsulated colorants that are said to provide a natural appearance when the cosmetic composition is applied to the skin. Polymer encapsulants are made from monomers such as styrene and methacrylates, which would provide opacity to conceal imperfections in the underlying skin.

The cosmetics manufacturing community desires alternatives to the use of optical barriers in order to mask skin imperfections. One alternative is disclosed in Japanese patent application JP2002020235 assigned to Asahi Glass Company, Ltd. This Japanese application discloses the use of hollow glass spherical particles having an average particle size not exceeding 25 microns in a cosmetic composition. The cosmetic composition is said to provide excellent softness, elasticity and texture.

There is a need by the cosmetics manufacturing community for further improvements in terms of cosmetics that will enhance the appearance of the skin without causing an opaque layer to form on the skin. The present invention provides one solution to that need.

### SUMMARY OF THE INVENTION

The present invention provides relatively small micron-sized polymer-coated glass ball lenses having optical characteristics that are uniquely suited for use in cosmetic compositions for topical application to the skin. The coated glass ball lenses are encapsulated within an outer protective coating of polymer having a refractive index of between about 1.25 and about 1.75. In one embodiment, the polymer has optical characteristics which are optionally identical, or nearly identical, to the optical characteristics of glass. The encapsulating polymer is preferably a polyurethane coating that optionally contains at least one of a colorant or dye, a contrast enhancer and a contrast inhibitor.

The encapsulating polymer coating may also contain a glass/polymer binding agent such as silane, for example, in the case of polyurethane. Illustrative silanes are the epoxy silanes, such as Dow Corning's Z-6040, which is a bifunctional silane containing a glycidoxy-reactive functional group and a trimethoxysilyl inorganic functional group. When employed in a cosmetic composition, the polymer-coated glass ball lenses serve to reduce or minimize the ability of the human eye to see skin imperfections such as blemishes and wrinkles while avoiding the formation of an opaque layer of cosmetic on the skin. The optional colorant or dye, contrast enhancer and contrast inhibitor serves to enhance the healthy appearance of the skin.

### BRIEF DESCRIPTION OF THE DRAWINGS

FIG. 1 is a cross sectional view of a polymer-encapsulated glass ball lens in accordance with the invention.

FIG. 2 is a schematic representation of a typical polymer-coated glass ball lens showing the collection and passage of ambient light through the lens when used in a cosmetic composition in accordance with the present invention.

### DESCRIPTION OF THE PREFERRED EMBODIMENTS

It has now been surprisingly found that skin appearance-enhancing polymer-coated clear, light-transmissive glass ball lenses can be provided for use in cosmetic applications. Without wishing to be bound by any particular theory, the present inventor believes that the coated glass ball lenses effectively serve as “double convex lenses”, that is, a lens arrangement similar to double capital letter “D” shaped lenses, one in reverse direction, placed together to form a sphere. In this “double convex lens” set, the lens effect is provided by the glass ball lens itself, and other effects are provided by the polymer coating on the outer surface of the glass ball lens. The polymer lens coating optionally serves as a vehicle for providing at least one of a dye or other colorant and an optics modifier such as a contrast enhancer or inhibitor that serves to enhance the appearance of the skin when employed in a cosmetics composition. The polymer lens coating optionally serves as a vehicle for the delivery of active materials such as skin moisturizers, vitamins such as Vitamin E, antimicrobial additives, other actives, and combinations of actives.

Thus, the present invention provides optical lenses and specifically coated ball lenses exhibiting a “double convex lens” characteristic, and optionally employs colorants and/or other visual modifiers to alter the optical characteristic of the skin to which a cosmetic containing the ball lenses is applied, as compared to skin without the cosmetic. The result is to effectively conceal aging, skin imperfections, blemishes, wrinkles, shadows, rashes, and the like. The optional colors, dyes and/or other visual modifiers are suitably employed within the outer encapsulating polymer coating that has a refractive index of between about 1.25 and about 1.75. In one embodiment, the polymer has a refractive index substantially equivalent to that of the glass core of the ball lenses. The polymer coating also serves to protect the skin from direct contact with the glass in the ball lenses, and further serves to enhance the “soft feel” to human touch of the texture associated with the ball lenses.

Although either solid or hollow glass ball lenses are suitably employed in the present invention, the ball lenses are preferably solid. Glass beads that are suitable for use in fabricating the coated ball lenses are described, for example, in U.S. Pat. No. 6,525,111, assigned to Prizmalite Industries Inc., the disclosure of which is incorporated herein by reference in its entirety.

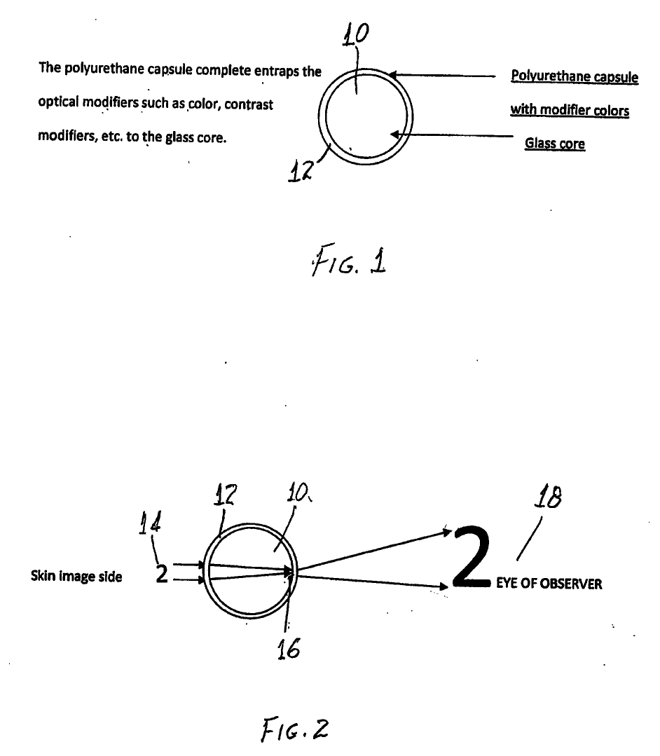

FIG. 1 shows a typical encapsulated ball lens mircosphere embodying the invention. As shown, the ball lens comprises a solid glass core 10 that is completely encapsulated within an outer protective polymer coating 12. The refractive index of the glass core 10 is about 1.51. The encapsulating polymer coating 12 has a refractive index between about 1.25 and about 1.75. Polyurethane is the preferred polymer for the coating 12, although other polymers such as polypropylene or polyester can also be employed, provided that the polymer's refractive index is between about 1.25 and about 1.75. Advantageously, the median diameter of the glass core 10 is no greater than 25 microns, preferably no greater than 15 microns, and more preferably no greater than 10 microns. The most preferable range for the diameter for the glass core 10 is between about 0.5 micron and about 10 microns. The encapsulating polymer coating has a thickness of between about 0.1 and 20 microns and may comprise from about 0.2% to about 25% by weight, and preferably from about 1% to about 20% by weight, of the total weight of the coated ball lens.

In one embodiment, the glass core 10 has a specific gravity of about 2.48, a radius of about 5.0 microns and a focal point of about 4.90 microns from the lens axis. Although the encapsulated ball lens is shown as having a solid core 10, it will be understood that the glass spheres may also be hollow and encapsulated within the same polymer coating 12.

The Ball Lens is a type of bidirectional biconvex lens that has been used extensively in analytical equipment and in the transfer of data within fiber optic systems. The focal point of the lens may be calculated from the following equation:

$$\frac{1}{f} = ( n - 1 ) [ \frac{1}{R 1} - \frac{1}{R 2} - + \frac{( n - 1 ) d}{nR 1 R 2} ]$$

wherein
f=Focal length of the lens
n=Refractive Index of the lens material
R1=Radius of the curvature of the lens surface closest the light source (or image)
R2=Radius of the curvature of the lens surface farthest from the light source (or image)
d=Is the thickness of the lens (the distance along the lens axis between the two surface vertices).

As shown in FIG. 2, the ball lens 10 focuses ambient light through the lens and thereby illuminates the nearby skin 14 with the modified ambient light emanating from the lens inward towards the skin, thus brightening the skin. Once illuminated, the image of the skin 14 is then passed back through the ball lens 10, through the focal point 16, where the lens 10 focuses the image of the skin toward the observer 18. The image 14 is actually magnified by the ball lens 10 but this is generally not noticed by the eye of the observer 18. The encapsulating coating 12 is embodied with optical modifiers that alter the light going into the skin and alter the image of the skin coming out to the eye of the observer 18. A key advantage of this configuration is the option to engineer colors into the encapsulating polymer coating 12 that change the human eye's ability to see skin imperfections. It seems not to matter to the casual observer viewing the image of the skin 14 through the ball lens 10 that the image is magnified since the optical and color modifiers that are employed impart a perspective to the image 14 that seems absent of most peripheral details such as depth and orientation. The advantage of the ball lens is that the focal point 16 is usually at or almost at the radial surface of the solid core 10. In the present case, the solid core radius is 5 microns, and the focal point is 4.9 microns in front of and equally behind the vertical axis.

The effect of the foregoing is that the image of the substrate skin 14 is a bright intense small disk emanating from the focal point 16, with the image enlarging or magnifying over distance from the focal point of the lens towards the viewer. The benefit of encapsulating the ball lens with polyurethane is that the coating 12 is optically close to the glass core 10 with image modifiers. The encapsulating material contains specific colors which minimize the ability of the eye to see skin imperfections, such as Green minimizing Rosacea, scarring, blemishes, while others minimize bruises, cellulite, and wrinkles. Bright red minimizes paleness and sallow color, creates the appearance of healthy “bloom”, and together with Green includes the band of colors that were once present in youthful skin and are now similarly restored to the skin image again to give the visual experience of a more youthful healthy person and help to minimize the appearance of the signs of aging including such things as wrinkles, uneven skin tone and a general lack of radiance. Contrast enhancers and modifiers such as transparent nano titanium dioxide and transparent nano zinc oxide, for example, create an altered depth of field impression to the eye of the observer, altering the experience of the shape of the face, as well as altering the clarity of detail. The cosmetic use of light and dark contrast enhancers and reducers cause the visual cortex to misinterpret the depth and flatness of individual features of the face. As indicated above, the glass ball lenses of the present invention may be solid or they may be hollow. In either case, they are completely encapsulated within a protective polymer coating having a refractive index nearly the same or close to that of glass, i.e., from about 1.25 to about 1.75. The encapsulating polymer coating is inert and non-reactive with other components of the cosmetic composition. Preferably, the polymer is also resistant to degradation at elevated temperatures of up to 120 degrees F. or more that may be encountered during processing of the microcapsules formulation into the cosmetic composition, and during storage and shipping prior to use of the cosmetic. The encapsulating polymer is advantageously resistant to degradation by water, solvents, and oils that may be present in cosmetic compositions. Colors, dyes and other optical modifiers that are optionally employed in the microcapsules of the present invention are advantageously fixed within the encapsulating polymer, and thus not free to pass into the skin when used in a cosmetic composition.

The encapsulated ball lenses of the invention are restricted in size to be smaller than the resolution of the human eye. Therefore, the encapsulated ball lenses are invisible to the eye and not perceivable as individual particles, while at the same time providing the optical effects of a ball lens.

The encapsulated ball lenses of the invention can be produced in a number of ways, the most effective being by use of a fluidized bed system. Using this system, the glass beads are fed into an air stream created as a vortex in which the air stream is the fluid bed that contains solids and liquids, which in this case are the ball lenses and the liquid water based polyurethane dispersion droplets, along with an optional volatile such as m-pyrol. The encapsulating polymer material may contain a glass/urethane binding agent such as a silane, in the case of polyurethane and may also incorporate a metal salt such as, for example calcium chloride (CaCl₂), magnesium chloride (MgCl₂), calcium sulfate, magnesium sulfate, sodium chloride, and mixtures thereof, which tends to further strengthen the resilience of the polymer coating. The preferred metal salt is calcium chloride. The glass particles are suspended in the air stream and are uniformerly coated with the polymer layer. The glass beads may be passed through the fluid bed containing the polymer along with an optional solvent, such as m-pyrol. The fluidized bed system helps avoid agglomeration of the glass beads until they are coated. The coated glass beads are dried to insure freedom from volatiles with little or no leaching of actives or dyes. After coating and drying, the polymer-coated beads are suitably collected in the form of a dry powder.

Optionally, colors and/or dyes may be used in the encapsulating coating to produce a number of desired visual perceptions in the eye of the observer when the coated particles are used in a cosmetic composition. The following are examplatory:
A red dye that reduces the appearance of pale or sallow skin and adds the “bloom of good health” to the applied areas.
A yellow dye that counteracts the appearance of slow deoxygenated blood beneath thin skin as below the eye, as well as bruises and circles under the eyes.
A green dye that provides the appearance of youthfulness to the skin and reduces the appearance of wrinkles.
A violet dye that lightens and brightens dull or swarthy skin.
A blue dye that enhances the whiteness or reduces the visual perception of skin pigmentation and produces skin lightening.
A visually clear untinted encapsulation to magnify the skin's visual presence and focus incoming ambient light.
Mixtures of dyes and particle sizes may also be employed to yield certain desirable effects. For example, smaller green particles can be mixed with an accepted red fluorescent dye larger particles with the casts pulled with transparent dye to make any ethnic blend needed. Yellow/green dye together with red makes brownish and red plus blue makes brown.

Generally speaking, the polymer coating may comprise from about 0.2% to about 25% by weight of the coated ball lenses and the color dyes, modifiers, etc. from about 0.001 to about 5.0% by weight of the composition. The cosmetic composition may also include a base medium, such as mineral or vegetable oil, solvents such as alcohol, water, perfumes, and other additives, as is well-known in the cosmetics industry.

The following example is intended to illustrate, but in no way limit the scope of, the present invention. All parts and percentages are by weight and all temperatures are degrees Celsius unless explicitly stated otherwise.

### EXAMPLE 1

### Method for Making Polymer-Coated Glass Ball lenses for Skin Image Modification

### Beaker Bath With an Homogenizer

Into a 500 ml beaker equipped with a homogenizer is charged 100 grams of water, followed by 75 grams of solid glass ball lenses having an average particle diameter of about 10 microns and a range of particle diameters of from 3 to 14 microns. The homogenizer is a high shear type, and the shear head was set to rotate at a speed of between about 1000 and 1600 rpms. The speed is adjusted to be sufficient to cause all of the glass ball lenses moving with none on the floor of the mixing vessel. The speed used depends upon the diameter of the head and the diameter of the beaker/vessel and the viscosity of the contents. The shear head is referred to as a “grapefruit” configuration, and is a one horsepower unit with speed AC controller from Arde Barenco.

The speed required is that which by observation shows that all the glass lenses are in active movement (swirling, not sinking to a stationery place on the bottom of the beaker). Once this has been stabilized as to movement, the polyurethane colloidal dispersion is charged into the beaker. The amount is based on the calculated weight to be between about 0.2% to about 25% polyurethane based on the weight of the balls lenses (e.g. 0.2% polyurethane and 99.8% glass spheres). The beaker is then anchored to the bench on top of an electric hot plate. Glass ball lens movement is maintained by homogenizer speed. The homogenizer needs to have a narrow head clearance to break agglomerations and prevent clumping. A suitable head clearance was found to be a clearance of 12-19 microns.

The colloidal dispersion of polyurethane used to encapsulate the glass ball lens is commercially available under the trademark SANCURE 847 dispersion, a product available from Noveon. Alternatively, other SANCURE products could have been used, such as those available under the product numbers 815, 835, 847, 1828 or 12954, or combinations thereof. The SANCURE 847 dispersion is an aliphatic polyester polyurethane solution containing water, polyurethane, amine, and N Methyl 2 pyrrolidinone. The aliphatic polyester polyurethane component has a number average molecular weight of approximately 50,000 and a weight average molecular weight of about 100,000. In preparing the colloidal dispersion, about 22.5 grams of polyurethane is dispersed under controlled conditions, and may be forced out of solution/dispersion by heating, the dropping of pH, as well as a controlled combination of the two factors. The polyurethane is colloidally dispersed and may be forced out of dispersion by destabilization controlled in the vortex on to the ball lenses. When the polyurethane is forced out of the water based solution it coats all hydrophobic materials, such as the glass ball lenses stirring in the solution, thus effectuating coating of the glass ball lenses.

Next, approximately 0.225 grams of D&C Green #5 dye was charged into the system in order to provide an amount of dye within a range of from 0.001% to 5% by weight based on the total weight of polyurethane solids in the system. Temperature in the beaker is still ambient. Ammonium sulfate is added at 8% on weight of the water and fully dissolved. This is included to promote the volatilization of the ammonia leaving sulfuric acid generated slowly by heating the beaker. Once this occurs, the heat is slowly ramped to 70° C. At this point a 1% solution of citric acid and a 1% solution of glycolic acid were introduced by very slow drip in different trials to lower the pH of the system, also promoting the forcing of the polyurethane out of dispersion and onto the ball lenses. Once all the polyurethane is forced out, calcium chloride was added to the bath to cross link the polyurethane capsule in place. It is believed that calcium chloride and heat cause the cross-linking of two carboxyl groups on adjacent polymer chains. The resulting polyurethane is tougher, more sealed and more impermeable to water, oils, soluble colorants, and most solvents, as compared to uncrosslinked polyurethane coatings.

The resulting product, namely the glass ball lens with polyurethane coating having a median coating layer thickness of about one micron and a range of thickness of between about 0.1 and 5 and containing dyes is then filtered through a 3 micron pore size silicone and fiberglass filter. Rinses are done with a light next to the vacuum Buchner funnel and filter using distilled water and continued until there is no color in the filtrate. The filter cake was then broken apart, and then force air dried in an oven at 120° F., making sure that the oven fan unit is on maximum flow, yield lowest clumping and the best handle. The resulting powder exhibited excellent optical characteristics including light transmission. The refractive index of the coated glass beads was 1.51

What is claimed is:

1. A composition for topical application to the skin comprising glass ball lenses, at least a portion of which are encapsulated within an outer coating of polymer having a refractive index of between about 1.25 and about 1.75.

2. The composition of claim 1 wherein the outer polymer coating contains at least one of a color, dye and visual modifier.

3. A composition according to claim 2, wherein the colorant or dye is selected from the group consisting of green, red, yellow, violet, blue, and mixtures thereof.

4. A composition according to claim 1 wherein the outer polymer coating has a refractive index substantially equivalent to that of glass.

5. A composition according to claim 1 wherein the glass ball lenses have a diameter of between about 0.5 and 25 microns, and wherein the outer polymer coating has a thickness of between about 0.1 and 20 microns, and wherein the polymer is selected from the group consisting of polyurethane, polypropylene, polyester and combinations thereof.

6. A composition according to claim 5, wherein the polymer is polyurethane.

7. A composition according to claim 5, wherein the outer polymer coating comprises from about 0.2% to about 25% by weight of the coated glass ball lenses.

8. A composition according to claim 7, wherein the outer polymer coating comprises from about 1 to about 20% by weight of the coated glass ball lenses.

9. A composition according to claim 5, wherein the outer polymer coating contains a glass/urethane binding agent.

10. A composition according to claim 9, wherein the glass/urethane binding agent is a silane.

11. A composition according to claim 5, wherein the outer polymer coating contains a metal salt to enhance the resilience of the polymer coating.

12. A composition according to claim 11, wherein the metal salt is calcium chloride.

13. A composition according to claim 1, wherein the encapsulated ball lenses are incorporated into a base medium comprising water or an oil in water emulsion to form a cosmetic composition.

14. A composition according to claim 5, wherein the glass ball lenses have a radius of about 5.0 microns and a focal point of about 4.9 microns measured from the lens axis.

15. A method for enhancing the appearance of skin comprising:
providing multiple glass ball lenses having Ball Lens characteristics,
encapsulating the ball lenses within an outer coating comprising a polymer having a refractive between about 1.25 and about 1.75, and
applying the encapsulated ball lenses to the skin.

16. A method according to claim 15, wherein the encapsulated ball lenses contain at least one of a color, dye and visual modifier.

17. A method according to claim 15, wherein the polymer is polyurethane.

18. A method according to claim 15, wherein the outer polymer coating contains a glass/urethane binding agent.

19. A method according to claim 18, wherein the binding agent is a silane.

20. A method according to claim 15, wherein the outer polymer coating contains a calcium salt to enhance the resiliency of the polymer coating.

21. A method according to claim 20, wherein the calcium salt is calcium chloride.

22. A ball lens comprising a glass core encapsulated within an outer coating comprised of a polymer having a refractive index between about 1.25 and 1.75, wherein the coating contains at least one of a color, dye and visual modifier.

23. The ball lens of claim 22, wherein said polymer is selected from the group consisting of polyurethane, polyester, polypropylene, and combinations thereof.
